(19) United States
(12) Patent Application Publication
(10) Pub. No.: US 2008/0300417 A1
(43) Pub. Date: Dec. 4, 2008

(54) Slurry reaction system

(76) Inventor: Te Chang, West Chester, PA (US)

Correspondence Address: LyondellBasell Industries, 3801 WEST CHESTER PIKE, NEWTOWN SQUARE, PA, 19073, US

(21) Appl. No.: 11/809,109

(22) Filed: May 31, 2007

(51) Int. Cl. C07D 301/04

(52) U.S. Cl. 549/523

(57) ABSTRACT

The present invention provides a slurry reaction system wherein liquid reaction mixture containing catalyst fines is separated from catalyst particles of a size suitable for use in the slurry reaction in a first separation step and in a subsequent separation step the catalyst fines are separated from liquid reaction mixture.

### BACKGROUND OF THE INVENTION

The present invention relates to an improved slurry reaction system such as that employed in the production of propylene oxide by reaction of oxygen, hydrogen and propylene in a liquid reaction medium such as a water and methanol mixture and employing a solid catalyst such as palladium promoted TS-1 slurried therein, and especially to a system wherein the reaction liquid slurry is first subjected to a separation treatment for removal of solid catalyst particles of relatively large size for recycle from liquid which contains reaction product as well as catalyst fines. The mixture containing product and fines is then subjected to further separation treatment for solid fines removal.

### DESCRIPTION OF THE PRIOR ART

Epoxides constitute an important class of chemical intermediates useful for the preparation of polyether polyols, glycols, glycol ethers, surfactants, functional fluids, fuel additives and the like. Many different methods for synthesizing epoxides from the corresponding olefins have been described in the literature. A Japanese patent application assigned to the Tosoh Corporation and published in 1992 (Kokai No. 4-352771) proposed making propylene oxide by reacting propylene, hydrogen and oxygen using a catalyst comprising a Group VIII metal and a crystalline titanosilicate. U.S. Pat. Nos. 6,281,369, 6,005,123 and 6,008,388 are also relevant. As with any chemical process, it would be desirable to attain further improvements in epoxidation methods of this type and it is to such improvements what this invention is directed.

A problem with prior systems has been the tendency for the slurried solid catalyst to undergo attrition due to agitation either by impellers and/or gases. The continuous generation and accumulation of fines within the slurry reactor causes problems in the separation of solids from liquids by filtration as the catalyst fines tend to plug or otherwise interfere with the separation procedures.

It is important in the slurry systems that the solid catalyst have a suitable size to function properly. Generally catalyst particles having an effective diameter of 10 to 100 microns, preferably 30 to 40 microns are suitable. Particles having an effective diameter below about 1 micron interfere with system operation.

### SUMMARY OF THE INVENTION

In accordance with the present invention, the slurry reaction mixture is subjected to a plurality of separation steps whereby the slurry is first treated to separate the relatively large size catalyst particles which are useful in the system from reaction liquid which also contains catalyst fines. The separated larger sized particles can conveniently be recycled to the reaction. The mixture of reaction liquid and fines is then treated in one or more filtration steps to separate solids free reaction liquid from the solids fines.

### DESCRIPTION OF THE DRAWINGS

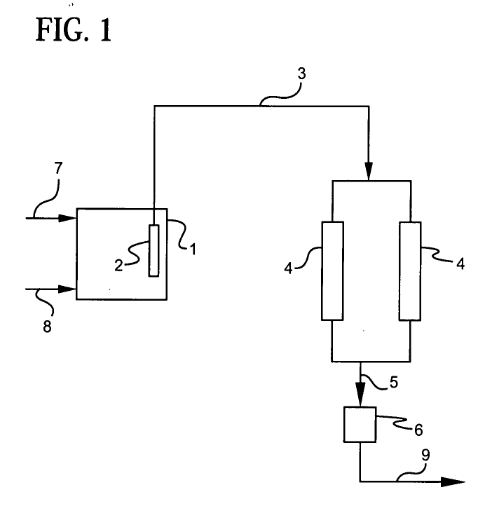

The accompanying FIG. 1 illustrates an embodiment of the invention wherein the first separation of larger sized particles is by means of an internal filter in the reactor.

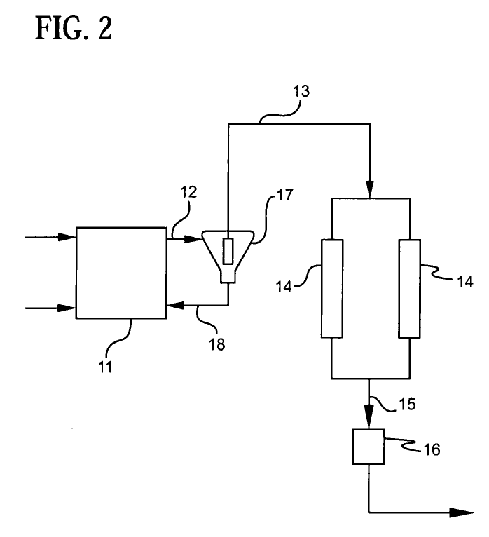

Accompanying FIG. 2 illustrates an embodiment wherein the first separation is by means of a filter system external to the reactor.

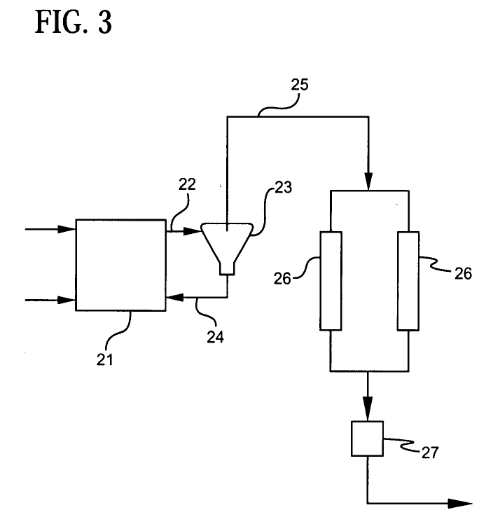

Accompanying FIG. 3 illustrates practice of the invention where separation of the large solids particles is by means other than filtration.

### DETAILED DESCRIPTION

Practice of the invention is especially advantageous in a system for the production of propylene oxide by reaction of propylene, oxygen and hydrogen in a liquid comprised of a solvent such as methanol or a methanol-water mixture and containing slurried noble metal promoted titanium silicalite.

The catalysts to be used in the present invention are comprised of a titanium or vanadium zeolite and a noble metal (preferably an element of Group VIII of the Periodic Table). Suitable zeolites are those crystalline materials having a porous molecular sieve structure with titanium or vanadium atoms substituted in the framework. The choice of zeolite will depend upon a number of factors, including the size and shape of the olefin to be epoxidized. For example, it is preferred to use a relatively small pore titanium or vanadium zeolite such as a titanium silicalite if the olefin is a lower aliphatic olefin such as ethylene, propylene, or 1-butene. Where the olefin is propylene, the use of a TS-1 titanium silicalite or vanadium silicalite is especially advantageous. For a bulky olefin such as cyclohexene, a larger port titanium zeolite such as a titanium zeolite having a structure isomorphous with zeolite beta may be preferred.

The titanium-containing zeolites useful as catalysts in the epoxidation step of the process comprise the class of zeolitic substances wherein titanium atoms are substituted for a portion of the silicon atoms in the lattice framework of a molecular sieve. Such substances are well known in the art.

Particularly preferred titanium-containing zeolites include the class of molecular sieves commonly referred to as titanium silicalites, particularly “TS-1” (having an MEL topology analogous to that of the ZSM-5 aluminosilicate zeolites), “TS-2” (having a MEL topology analogous to that of the ZSM-11 aluminosilicate zeolites), and “TS-3” (as described in Belgian Pat. No. 1,001,038). Also suitable for use are the titanium-containing molecular sieves having framework structures, isomorphous to zeolite beta, mordenite, ZSM-48, ZSM-12 and MCM-41. The titanium-containing zeolite preferably contains no elements other than titanium, silicon and oxygen in the lattice framework, although minor amounts of boron, iron, aluminum, and the like may be present. Other metals such as tin or vanadium may also be present in the lattice framework of the zeolite in addition to the titanium as described in U.S. Pat. Nos. 5,780,654 and 5,744,619.

Preferred titanium-containing zeolite catalysts suitable for use in the process of this invention will generally have a composition corresponding to the following empirical formula: xTiO₂(1-x)(SiO₂), where x is between 0.0001 and 0.500. More preferably, the value of x is from 0.01 to 0.125. The molar ratio of Si:Ti in the lattice framework of the zeolite is advantageously from 9.5:1 to 99:1 (most preferably from 9.5:1 to 60:1). The use of relatively titanium-rich zeolites may also be desirable.

While any of the noble metals can be utilized (i.e., gold, silver, platinum, palladium, iridium, ruthenium, osmium), either alone or in combination, palladium is particularly desirable. Typically, the amount of noble metal present in the catalyst will be in the range of from 0.01 to 20 weight percent, preferably 0.1 to 5 weight percent. Suitable catalysts are described in U.S. Pat. Nos. 6,281,369, 6,005,123 and 6,008,388 the disclosure of which are incorporated herein by reference.

The amount of catalyst used may be determined on the basis of the molar ratio of the titanium contained in the titanium zeolite to the olefin that is supplied per unit of time. Typically, sufficient catalyst is present to provide a feed ratio of from about 10 to 100,000 lbs of propylene/hr per lb of titanium.

Referring to FIG. 1, conventional slurry reactor 1 is provided for the production of propylene oxide. See U.S. Pat. No. 6,376,686. Located within reactor 1 is internal filter 2 which is sized to retain within reactor 1 solid catalyst particles which are effective as slurried catalyst for propylene oxide production, suitable particles having an effective diameter of at least 10⁻³ mm, preferably 3×10⁻³ to 4×10⁻³ mm, while permitting passage through the filter of reaction liquid and catalyst fines, i.e. particles of less than 10⁻⁴ mm average particle size.

The reaction liquid and fines admixture passes via line 3 to secondary filtration units 4 which effectively filter out fines from the reaction liquid. The reaction liquid essentially free of solids passes via line 5 to tertiary filter system 6 for further removal of fines. In many cases, the tertiary filtration step can be omitted as solids separation is effectively complete with filtration means 2 and 4.

The product containing reaction liquid passes from filtration unit 6 via line 9 for further treatment and product recovery (not shown).

As shown in FIG. 1, the various reaction feed materials are introduced via lines 7 and 8 each of which may be a plurality of separate lines. Agitation is provided by an impeller (not shown) or by the introduced vapor or by a combination of these.

In accordance with this embodiment, filter 2 is sized to effectively retain the appropriate sized catalyst particles in the reaction liquid in reactor 1 while removing liquid reaction mixture and catalyst fines. The fines are separated from reaction liquid in the filtration units 4 and 6 by conventional operation.

A somewhat different embodiment of the invention is shown in attached FIG. 2. In this embodiment, reaction liquid containing both proper sized catalyst particles as well as catalyst fines is removed from the reactor 11 and passes via line 12 to external filtration unit 17 which is sized to retain the appropriate size catalyst particles having at least 10⁻³ mm effective diameter which are then recycled via line 18 to the reaction. Liquid containing catalyst fines is then passed via line 13 and treated essentially as described in connection with FIG. 1. Fines are separated in filtration units 14 and, if necessary, in filtration unit 16.

Generally speaking, both the internal and external filters are sized for a liquid flow or flux of about 0.01 to about 2 gallons per minute per square foot. The filtration step is operated at a differential pressure of about 0 to about 120 psia.

FIG. 3 describes practice of the invention where separation means other than filtration means, located external to the reactor, are used to separate the larger catalyst particles for recycle from the mixture of reaction liquid and catalyst fines. As shown in FIG. 3, reaction liquid containing both normal sized catalyst particles as well as catalyst fines is removed from reactor 21 and passed via line 22 to settler 23.

In the settler the larger particles are separated and returned via line 24 to reactor 21. Reaction liquid containing catalyst fines is passed via line 25 to filters 26 and 27 for final separation of fines as described above.

In place of the settler, equivalent separating means such as hydroclones or elutriators can be used.

The following examples illustrate the invention.

### EXAMPLE 1

Referring to FIG. 1, a feed gas mixture comprised by volume of 5% H₂, 10% oxygen, 15% propylene, balance methane, is fed to agitated slurry reactor 1 via line 7. A slurry of palladium promoted TS-1 (0.1 wt. % Pd) in methanol/water is retained in the reactor with a 5 micron internal filter 2. The catalyst particle size as added is about 30 to 40 micron diameter. Suitable agitations (not shown) are provided to continuously maintain a slurry of catalyst particles in the liquid reaction mixture while feeding methanol/water continuously via line 8.

The reaction is carried out at 60° C. and 300 psig with a residence time in the reactor of 2-4 hours. In the course of reaction with agitation and gas sparging over 2 months a substantial portion of the catalyst particles are reduced in particle size by attrition to finer particles of 1 micron diameter or less at a rate of about 0.14% per day.

Means are provided in reactor 1 in the form of a slurry draw-off for continuous removal of reaction product containing liquid. Surrounding the slurry draw-off is filter screen 2 which is sized to permit passage of liquid containing the catalyst fines while retaining particles of 1 micron diameter or greater in reactor 1. Screen 2 is appropriately 5 micron or finer and is operated at a liquid flux or flow rate of 0.15 gpm/sq.ft. Filter 2 is backwashed with solvent feed when filter differential pressure reaches 80 psia.

Reaction liquid containing catalyst fines passes via line 3 to filters 4 which are operated alternately and which effectively retain catalyst fines which permitting passage of reaction liquid. The reaction liquid passes via line 5 to finishing filter 6 wherein fines separation is completed. In many cases, filter 6 is not needed as the filtration is completed in filters 4.

From filter 6, the reaction liquid passes via line 10 to product separation and solvent recycle which are carried out in accordance with normal practice.

As indicated above, solvent and make-up catalyst are added as needed via line 8.

### EXAMPLE 2

Referring to FIG. 2 operation is analogous to that described in FIG. 1 except that the reaction mixture and catalyst slurry is removed from the reactor 11 via withdrawal line 12 and is passed to filter 17 which is adapted to separate catalyst particles having diameter of 1 micron or greater and return these separated solids along with solvent via line 18 to the reactor.

Reactor mixture with contained catalyst fines is passed via line 13 to filters 14 where it is treated as above described in Example 1.

Practice of the invention is especially advantageous in that continuous operation of the system is achieved without the difficulties caused by the accumulation of fines as contrasted with prior practices.

1. (canceled)

2. A process for producing propylene oxide by reaction of propylene, oxygen and hydrogen in a slurry of catalyst particles having an average diameter of 30 to 40 micron and comprising a titanium or vanadium zeolite and a noble metal in a solvent, wherein a substantial portion of the catalyst particles are reduced in particle size by attrition to finer particles of less than 1 micron diameter, the improvement wherein in a first separation step catalyst particles of at least 5 micron diameter are separated from reaction liquid containing catalyst fines having less than 1 micron diameter, and in a subsequent separate separation the catalyst fines are filtered from reaction liquid.

3. The process of claim 2 wherein said first separation step comprises an internal or external filtration and the liquid flow rate through the filtration is 0.01 to 2 gpm/sq.ft.

4. The process of claim 2 wherein the filtration is operated at a differential pressure of 0 to 120 psia.

5. The process of claim 2 wherein the catalyst particles comprise noble metal promoted titanium silicalite.

6. The process of claim 2 wherein the solvent is methanol or methanol-water.
